(19) United States
(12) Patent Application Publication
(10) Pub. No.: US 2006/0275321 A1
(43) Pub. Date: Dec. 7, 2006

(54) Polypeptide antagonists of HIV-1 Tat protein

(75) Inventor: Jaw-Kang Chang, San Carlos, CA (US)

Correspondence Address: CR MILES, P.C., 1 OLD TOWN SQUARE, SUITE 200 B, FORT COLLINS, CO, 80524, US

(73) Assignee: Phoenix Pharmaceuticals, Inc.

(21) Appl. No.: 11/143,550

(22) Filed: Jun. 1, 2005

(51) Int. Cl. A61K 31/70; A61K 39/12; A01N 43/04

(52) U.S. Cl. 424/204.1; 514/44

(57) ABSTRACT

Compositions and methods of using such compositions to regulate biological responses associated with binding of HIV-1 polypeptides.

### I. BACKGROUND

Compositions and methods of using such compositions to regulate biological responses associated with binding of HIV-1 polypeptides.

The human immunodeficiency virus type 1 (HIV-1) Tat gene product trans-activates viral gene expression and is essential for HIV-1 replication. The Tat protein exists in two forms, which in the HXB 2 viral isolate consists of 72 and 86 amino acids (“HIV-1 Tat polypeptides”). The 86-residue protein is encoded by two exons, whereas the 72-residue protein, which is identical except for lacking the 14 C-terminal residues is the product of the first Tat exon and is in itself sufficient for trans activation of viral gene expression. See FIGS. 1A and 1B.

Both the 86 residue and 72 residue HIV-1 Tat polypeptides appear to regulate a variety of intracellular responses including, but not limited to, the production of interferon and the subsequent cascade of events leading to inhibition of protein synthesis; bind a variety of cellular factors, including, but not limited to HIV long terminal repeat (LTR) RNA trans-activation response (TAR) element region, a putative ATPase and DNA helicase, a 36-kDA nuclear factor, as well as the transcriptional factors FFIID and Sp11; affect neurotransmitter release including, but not limited to, the release of acetylcholine; induce neurological impairments and neurotoxicity by mechanisms involving Ca²⁺ homeostasis after binding and depolarizing neuronal membranes.

There appears to be a large commercial market for molecular structures which target the binding sites or regulate intracellular responses associated with the intact HIV-1 Tat polypeptides. Because of the growing commercial, therapeutic, and research potential for such molecular structures, numerous research studies have been conducted which disclose a variety of uses for whole HIV-1 Tat polypeptides. In spite of the numerous studies conducted with whole HIV-1 Tat polypeptides, substantial problems remain unresolved with regard to providing molecular structures that can be commercialized, used as drug therapies in humans, or in research which target the binding sites and regulate intracellular responses associated with intact whole HIV-1 Tat polypeptides.

A first substantial impediment in developing molecular structures which regulate the intracellular processes associated with HIV-1 Tat polypeptides can be the difficulty in determining which residues encompassed by the primary structures of the 86 and 72 forms of the HIV-1 Tat polypeptides may be responsible for regulation of the above-mentioned intracellular responses. One aspect of this difficulty may be that there are multiple mechanisms for viral activation by the Tat proteins: a TAR-dependent transactivation of HIV-LRT, and a TAR-independent activation of virus replication involving the host factor NF-κB. See for example, Boykins et al., Cutting Edge: A Short Polypeptide Domain of HIV-1-Tat Protein Mediates Pathogenesis, The Journal of Immunology, 163:15-20 (1999). As to the first mechanism, it may be that residues between about 20 and 45 are implicated, while in the second mechanism the residues between about 73 and 86 appear to be implicated. See for example, Brand et al., The Tat Protein of Human Immunodeficiency Virus Type 1 Is a Substrate and Inhibitor of the Interferon-Induced, Virally Activated Protein Kinase, PKR, Journal of Biological Chemistry, Vol. 272, No. 13 (March 1997).

Another substantial problem in developing molecular structures which regulate the intracellular processes associated with HIV-1 Tat polypeptides can be that certain regions of the primary structure of the protein must be held in a specific secondary or tertiary structure by the remaining portions of the protein molecule to acquire biological activity. In this regard, the primary structure of the HIV-1 Tat polypeptides includes seven cysteine residues between position 22 and position 37 of the primary sequence which may yield various isomers of intact HIV-1 Tat polypeptides which may include isomers which have no disulfide bridge or isomers which include between one to three disulfide bridges implicating various combinations and permutations of the available free sulfhydryls.

Another substantial problem in developing molecular structures which regulate the intracellular processes associated with HIV-1 Tat polypeptides can be that certain residues must be phosphorylated to generate certain biological activities. Binding studies suggest that a correlation between phosphorylation and the ability of HIV-1 Tat polypeptides to bind double stranded RNA-activated kinase. In the 86 residue HIV-1 Tat, it appears that residues 49-72 may be important for phosphorylation while in the 72 residue HIV-1 Tat it appears that residues 62, 64 and 68 may be important.

Another substantial problem in developing molecular structures which regulate the intracellular processes associated with HIV-1 Tat polypeptides can be that a portion of an HIV-1 polypeptide when chemically enzymatically excised, or when identified and subsequently chemically synthesized, may not be biologically available to HIV-1 polypeptide target receptors in-vitro or in-vivo. This lack of biological availability may be due to insolubility of the compound, a binding affinity to surrounding substrates that is greater than to the target cell receptor, instability of the excised or chemically synthesized peptide with respect to cleavage, or with respect to modification of the peptide backbone, N-terminus, C-terminus, side chain, or other peptide or chemical moiety associated with the excised or chemically synthesized portion of the protein. Due to these, and a variety of other difficulties well known to those with skill in the art, assignment of biological activity to any specific biochemical structure, which may be a portion of a protein, such as the HIV-1 polypeptides, or any other molecule, may be unpredictable without an actual reduction to practice involving at least isolation, purification, and in-vitro assays to confirm biological activity of a particular compound.

Another substantial problem in developing molecular structures which regulate the intracellular processes associated with HIV-1 Tat polypeptides can be that it is difficult to produce sufficient amounts for wide spread use either for research or for therapies. As above-discussed the primary sequence of the HIV-1 Tat polypeptides can yield a large number of isomers due to differences in phosphorylation and disulfide bridge formation (or the lack thereof). To limit the number of isomers of the HIV-1 Tat polypeptide, excised fragments, or chemically synthesized peptides encompassing all or a portion of the HIV-1 Tat polypeptides, a method of generating or purifying single isomers of HIV-1 Tat polypeptides in sufficient quantity for commercial, therapeutic, or research use must be developed.

Another significant problem with conventional HIV-1 Tat polypeptides or HIV-1 polypeptide fragments may be that biological activity may insufficient for therapeutic use. One aspect of high dosages with respect to conventional peptide therapies may be that a substantial portion of the protein or peptide by weight does not contribute to the observed biological activity. A second aspect of high dosages with respect to conventional peptide therapies may be that the biological activity on a molar basis may be lower than is practical for a particular application. A third aspect of high dosages with respect to conventional peptide therapies may be that HIV-1 Tat polypeptides are unstable with respect to proteolytic activity, temperature, handling, or methods of in-vitro or in-vivo assays, or may have other attributes such as insolubility, a processing requirement, or high elimination rates in-vivo, as examples, which may render the active portions of conventional peptide dosage biologically unavailable or at levels which are not practical for applications such as human drug therapy.

With respect to making and using molecular structures to regulate or control biological responses associated with HIV-1 polypeptides, the present invention discloses methods of making and using a numerous and wide variety of molecular structures which address each of the above-mentioned problems.

### II. SUMMARY OF THE INVENTION

Accordingly, a broad object of the invention can be to provide compositions which can be used to regulate biological responses associated with HIV-1 polypeptides. One aspect of this broad object of the invention can be to provide compositions which can be used as antagonists to compete or prevent binding of HIV-1 polypeptides to specific targets to inhibit, reduce, alter, or otherwise control biological responses to HIV-polypeptides.

Another broad object of the invention can be to identify a method of chemical synthesis for the production of compositions which bind specific targets of HIV1-polypeptides. One aspect of this object of the invention can be to identify a method of chemical synthesis of peptides having contiguous amino acids identical to all or a portion of the HIV1-polypeptide sequence which allows selective disulfide bridge formation between a pair or pairs of cysteine residues in the synthesized peptides.

Another broad object of the invention can be to provide compositions which can be used as antigens for the production of monoclonal or polyclonal antibodies which bind HIV-1 polypeptides in-vivo.

Another broad object of the invention can be to provide compositions which bind target molecules of HIV-1 polypeptides resulting in or as antagonists to HIV-polypeptide responses such as inhibition of protein synthesis such as HIV long terminal repeat (LTR) RNA trans-activation response (TAR) element region, putative ATPase and DNA helicase, 36-kDA nuclear factor, as well as the transcriptional factors FFIID and Sp11; or affect neurotransmitter release including, but not limited to, the release of acetylcholine; or induce neurological impairments and neurotoxicity by mechanisms involving Ca²⁺ homeostasis after binding and depolarizing neuronal membranes.

Another broad object of the invention can be to provide compositions which can be used as carrier molecules to transfer otherwise impermeable compounds across the cell membrane and affect translocation of such compounds to target location within the cell.

Another broad object of the invention can be to provide compositions which mobilize intracellular calcium ion. One aspect of this broad object can be to mobilize calcium ion from the endoplasmic reticulum, acid filled calcium stores, or from tharsigargin-sensitive or tharsigargin-insensitive stores. Another aspect of this broad object of the invention can be to open voltage-gated calcium channels.

Another broad object of the invention can be to provide compositions which are antagonists to calcium mobilization in the treatment of vascular disorders such as hypertension or stroke; or for use in the treatment of HIV-1 dementia or other HIV-1 neurological disorders; or in the treatment of Alzheimer's Disease or other dementia disorders.

Naturally, further objects of the invention are disclosed throughout other areas of the specification, drawings, and claims.

### III. A BRIEF DESCRIPTION OF THE DRAWINGS

FIG. 1A is the primary sequence of the HIV-1 Tat eight six residue polypeptide (HIV-Tat 86).

FIG. 1B is the primary sequence of the HIV-1 Tat seventy two residue polypeptide (HIV-Tat 72).

FIG. 1C is an embodiment of the invention including the N-terminal portion polypeptide generated by cleavage of the HIV-Tat 86 or HIV-Tat 72 at the dibasic residue pair Lysine 28-Lysine 29.

FIG. 1D is an embodiment of the invention including C-terminal portion polypeptide generated by cleavage of HIV-Tat 86 at the dibasic residue pair Lysine 28-Lysine 29.

FIG. 2A is an embodiment of the invention including the primary sequence of HIV-1 30-86.

FIG. 2B illustrates a combined BOC and FMOC synthesis strategy to generate the primary sequence of HIV-1 30-86 of FIG. 2A using S-acetamidomethyl-L-cysteine (Cys-Acm) at residues 30 and 34 and S-Trt-L-cysteine at residues 31 and 37.

FIG. 2C is an embodiment of the invention including a peptide cleavage product which results from treatment of the protected BOC and FMOC synthesis product of FIG. 2B with hydrogen fluoride.

FIG. 2D is an embodiment of the invention including a peptide product which results from I₂/HOAc treatment of the peptide cleavage product of FIG. 2C.

FIG. 2E is an embodiment of the invention including a peptide product which results from further treatment of the peptide of FIG. 2D with iodine in HOAc.

FIG. 3A is an embodiment of the invention HIV-1 30-57 which can be chemically synthesized in a manner similar to that shown by FIG. 2B.

FIG. 3B is an embodiment of the invention including HIV-1 30-57 having a disulfide bridge between residue 31 and residue 37.

FIG. 3C is an embodiment of the invention including HIV-1 30-57 having a pair of disulfide bridges with a first between residue 31 and residue 37 and a second between residue 30 and residue 34.

FIG. 3D is an embodiment of the invention including HIV-1 30-57 having a pair of disulfide bridges with a first between residue 30 and residue 31 and a second between residue 34 and residue 37.

FIG. 3E is an embodiment of the invention including HIV-1 30-57 having a pair of disulfide bridges with a first between residue 30 and residue 37 and a second between residue 31 and residue 34.

FIG. 3F is an embodiment of the invention including HIV-1 32-57 which can be chemically synthesized in a manner similar to that shown by FIG. 2B.

FIG. 3G is an embodiment of the invention including HIV-1 32-57 having a disulfide bridge between residue 34 and residue 37.

FIG. 3H is an embodiment of the invention including HIV-1 34-57 which can be chemically synthesized in a manner similar to that shown by FIG. 2B.

FIG. 3I is an embodiment of the invention including HIV-1 34-57 having a disulfide bridge between residue 34 and residue 37.

FIG. 4A is an embodiment of the invention including HIV-1 48-86 which can be chemically synthesized in a manner similar to that shown by FIG. 2B.

FIG. 4B is an embodiment of the invention including HIV-1 48-84 which can be chemically synthesized in a manner similar to that shown by FIG. 2B.

FIG. 4C is an embodiment of the invention including HIV-1 58-86 which can be chemically synthesized in a manner similar to that shown by FIG. 2B.

FIG. 4D is an embodiment of the invention including HIV-1 62-86 which can be chemically synthesized in a manner similar to that shown by FIG. 2B.

FIG. 4E is an embodiment of the invention including HIV-1 61-80 which can be chemically synthesized in a manner similar to that shown by FIG. 2B.

FIG. 4F is an embodiment of the invention including HIV-1 49-57 which can be chemically synthesized in a manner similar to that shown by FIG. 2B.

FIG. 5A is a particular embodiment of the invention HIV-1 32-62 having a disulfide between residue 34 and residue 37 which functions to regulate intracellular responses associated with intact HIV-1 Tat protein.

FIG. 5B is a particular embodiment of a first peptide region of the invention HIV-1 49-57 which functions to transfer embodiments of the invention across the cell membrane.

FIG. 5C is a particular embodiment of a second peptide region of the invention HIV-1 32-48 having a disulfide bridge between residue 34 and residue 37 which functions to regulate intracellular responses associated with intact HIV-1 Tat protein.

FIG. 5D is an alternate embodiment of first peptide region illustrated by FIG. 5B.

FIG. 5E is an alternate embodiment of first peptide region illustrated by FIG. 5B.

FIG. 5F is an alternate embodiment of first peptide region illustrated by FIG. 5B.

FIG. 5G is an alternate embodiment of first peptide region illustrated by FIG. 5B.

FIG. 5H is an alternate embodiment of first peptide region illustrated by FIG. 5B.

FIG. 5I is an alternate embodiment of first peptide region illustrated by FIG. 5B.

FIG. 5J is an alternate embodiment of first peptide region illustrated by FIG. 5B.

FIG. 5K is an alternate embodiment of first peptide region illustrated by FIG. 5B.

FIG. 5L is an alternate embodiment of first peptide region illustrated by FIG. 5B.

FIG. 5M is an alternate embodiment of first peptide region illustrated by FIG. 5B.

FIG. 5N is an alternate embodiment of first peptide region illustrated by FIG. 5B.

FIG. 5O is an alternate embodiment of first peptide region illustrated by FIG. 5B.

FIG. 5P is an alternate embodiment of first peptide region illustrated by FIG. 5B.

FIG. 6A is a particular embodiment of the invention which includes an isolated biologically active region of the HIV-1 Tat polypeptides including HIV-1 30-57

FIG. 6B is a particular embodiment of the invention which includes an isolated biologically active region of the HIV-1 Tat polypeptides including HIV-1 31-57.

FIG. 6C is a particular embodiment of the invention which includes an isolated biologically active region of the HIV-1 Tat polypeptides including HIV-1 32-57.

FIG. 6D is a particular embodiment of the invention which includes an isolated biologically active region of the HIV-1 Tat polypeptides including HIV-1 33-57.

FIG. 6E is a particular embodiment of the invention which includes an isolated biologically active region of the HIV-1 Tat polypeptides including HIV-1 34-57.

### IV. DETAILED DESCRIPTION OF THE PREFERRED EMBODIMENTS

As can be easily understood from the foregoing, the basic concepts of the present invention may be embodied in a variety of ways. It involves a family of purified polypeptides modeled from the human immunodeficiency virus type 1 (HIV-1) Tat gene products (86 and 72 residues as above described)(hereinafter “HIV-1 Tat polypeptides”) including peptide fragments of the HIV-1 Tat polypeptides and polypeptides which include disulfide bridges or other types of bridges, linkages, or bonds between cysteine residues in various permutations and combinations and which can further include a peptide region which facilitates transfer of such polypeptide(s) across the cell membrane, and methods of producing such purified polypeptides, which can be utilized as or to facilitate transfer of membrane impermeable compounds into the cell; transfer such impermeable compounds to target sites within the cell; mobilize intracellular calcium ion including but not limited to calcium from the endoplasmic reticulum, tharsigargin-sensitive and tharsigargin-insensitives stores, acid filled calcium stores; activate calcium-induced calcium release; open voltage-gated calcium channels; HIV-1 tat antagonists; calcium mobilization antagonists; treat vascular disorders, stroke, or hypertension; treat HIV-1 dementia Alzheimer's Disease, or other dementia or neurological disorders; treat elevated or low calcium levels; or the like.

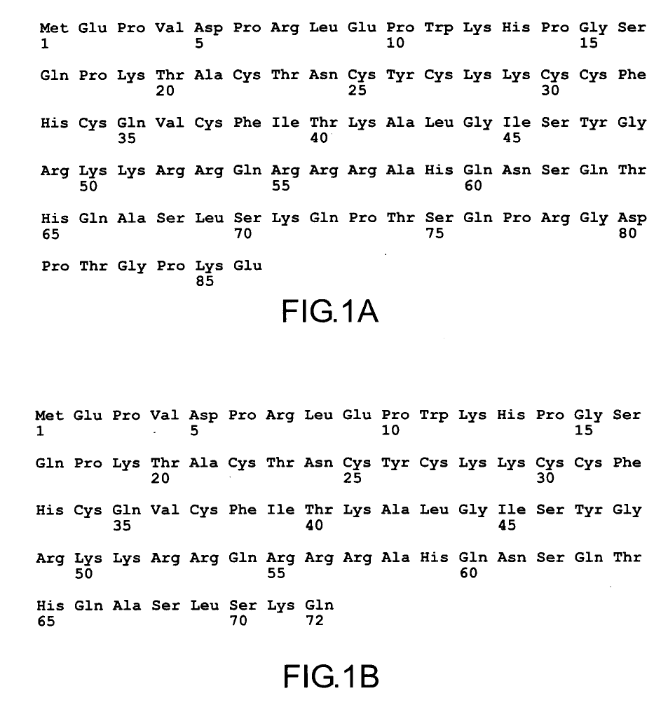

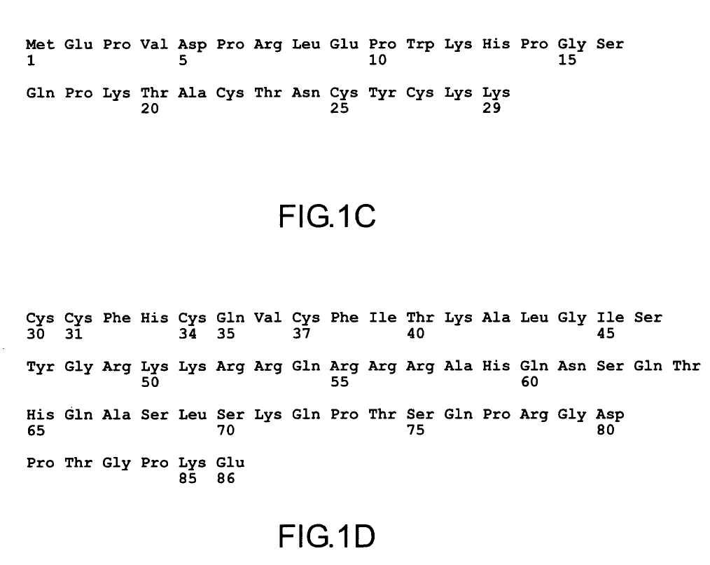

The HIV-1 Tat polypeptides comprise the HIV-1 Tat eighty six residue polypeptide (HIV-Tat 86) (SEQ ID NO: 1) as shown by FIG. 1A and the HIV-1 Tat seventy two residue polypeptide (HIV-Tat 72) as shown by FIG. 1B which are identical except for lacking 14 residues from the C-terminus. The HIV-1 Tat polypeptides may be processed in-vivo at the dibasic residue pair Lys-Lys 28-29 (K-K 28-29) which upon proteolytic cleavage can generate a N-terminal portion polypeptide as shown by FIG. 1C and a C-terminal polypeptide of the HIV-1 Tat polypeptides as shown by FIG. 1D.

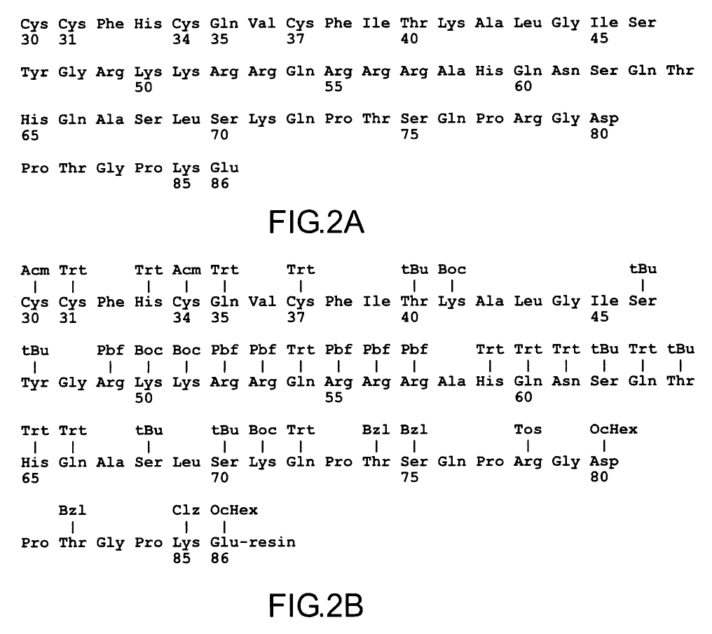

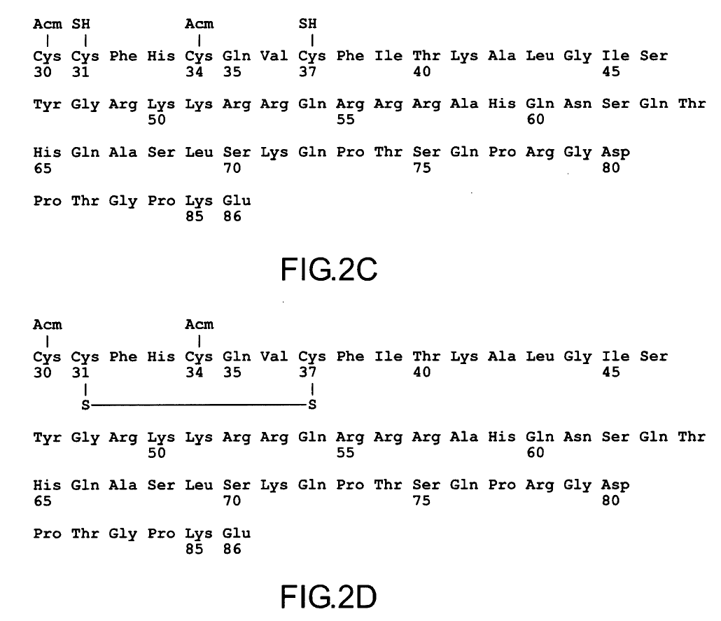

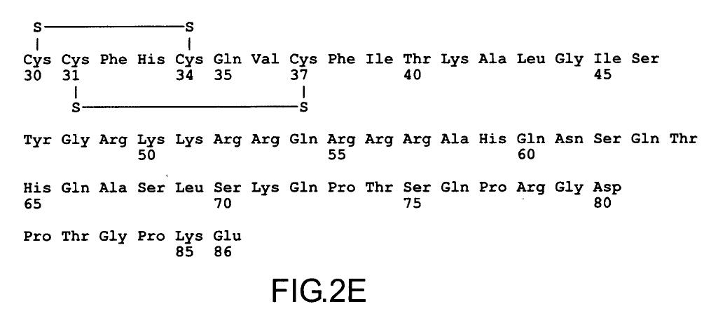

Now referring to FIG. 2, a polypeptide modeled after the C-terminal portion peptide can comprise residues 30-86 of the HIV-Tat 86 (HIV-1 30-86)(SEQ ID NO.: 7) as shown by FIG. 2A. The HIV-1 30-86 polypeptide can be chemically synthesized by the solid phase method using BOC-Glu(OcHex)-resin (0.6 mM/g, 0.67 g total 0.2 mM) as the solid support and by addition of fluorenyloxymethylcarbonyl (FMOC) amino acids or tertbutyloxymethylcarbonyl (BOC) amino acids with either an automated peptide synthesizer or manually using FMOC or BOC synthesis techniques well known to those skilled in the art. Naturally, other peptide synthesis strategies can be used to generate the polypeptides described herein and any specific synthesis strategy is intended to be illustrative rather than limiting with regard to the manner of making and using the various embodiments of the invention. See also, Solid Phase Peptide Synthesis: A practical approach, E. Atherton and R. C. Sheppard, IRL Press, Oxford, England, hereby incorporated by reference.

Specifically with respect to the HIV-1 30-86 polypeptide (SEQ ID NO.: 7) as a non-limiting illustrative example, S-acetamidomethyl-L-cysteine (Cys-Acm) can be incorporated as residue 30 and as residue 34 and S-Trt-L-cysteine can be incorporated as residue 31 and as residue 37 during chemical synthesis when an FMOC synthesis strategy is employed for the addition of amino acid residues as shown by FIG. 2B. Hydrogen fluoride cleavage of the protected HIV-1 30-86-resin including these S-protected cysteine residues yields a crude HIV-1 30-86 with S-acetamidomethyl-L-cysteine (Cys-Acm) residues at positions 30 and 34 and Cys-SH residues at positions 31 and 37 (SEQ ID NO.: 12) as shown by FIG. 2C.

The resulting mixture of polypeptides from the chemical synthesis of HIV-1 30-86 as above-described can be separated from one another by reverse phase HPLC using columns packed with silica having a pore of between 80 Δ and 300 Δ and a C-4, C-8, or C-18 ligand attached. The columns can be equilibrated with 0.1% trifluoroacetic acid in water at a flow rate dependent on column size as would be understood by those of ordinary skill in the art. The synthetic peptide mixtures can be applied to the reverse phase HPLC columns and eluted with 0.1% trifluoroacetic acid in acetonitrile using a gradient of 0% to about 80% over a period of about 30 minutes to about 1 hour. Fractions were collected at about 0.5 minute intervals. Fractions were subsequently analyzed for homogeneity by re-application and elution from the reverse phase HPLC system, mass spectrometry, SDS-PAGE, or automated Edman degradation on an Applied Biosystems Model 470A protein sequencer. As described by Applied Biosystems, Inc., Foster City, Calif.

Purification by HPLC as above-described can result in an amount of HIV-1 30-86 Cys-Acm 30-34 peptide (SEQ ID NO.: 12) as shown by FIG. 2C of sufficient quantity (about 16 milligrams with respect to the above-described synthesis and purification) and purity to be introduced into assays as described below or further treated with I₂/HOAc to form a disulfide bridge between Cys 31 and Cys 37 (Cys 31-Cys 37) (SEQ ID NO.: 13) as shown by FIG. 2D. Again purification by HPLC, as above-described, can result in an amount of HIV-1 30-86 Cys-Acm 30 and Cys-Acm 34 and Cys 31-Cys 37 (SEQ ID NO.: 13) which can be introduced into assays as described below or can be further treated with iodine in HOAc (7.5 mL and 25 mL respectively) to form a second disulfide bridge between Cys 30 and Cys 34 resulting in HIV-1 30-86 Cys 30-Cys 34 and Cys 31-Cys 37 (SEQ ID NO.: 9) as shown by FIG. 2E. HIV-1 30-86 Cys 30-Cys 34 and Cys 31-Cys 37 (SEQ ID NO.: 9) can again be HPLC purified as above-described to yield a purified HIV-1 30-86 Cys 30-Cys 34 and Cys 31-Cys 37 (SEQ ID NO.: 9)(about 3.05 mg in the above-described synthesis and purification) which presents a single significant peak on chromatograms when analyzed utilizing analytical HPLC and generates a single molecular ion at 6506.1 (M. W. 6505.42). Reduction of the disulfide bridges at Cys 30-Cys 34 and Cys 31-Cys 37, or other disulfide bridges with a reductant such as dithiothreitol, can result in the linear free sulfydryl peptide such as HIV 1-30-86 peptide (SEQ ID. NO: 7) as shown by FIG. 2A.

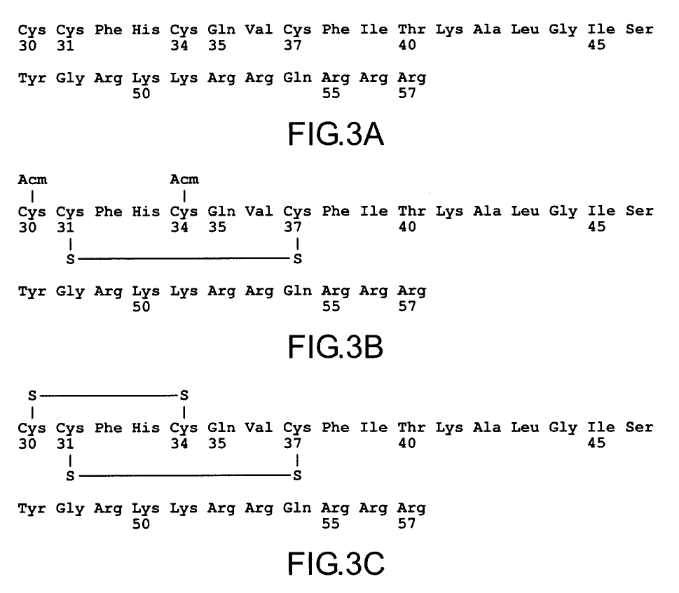

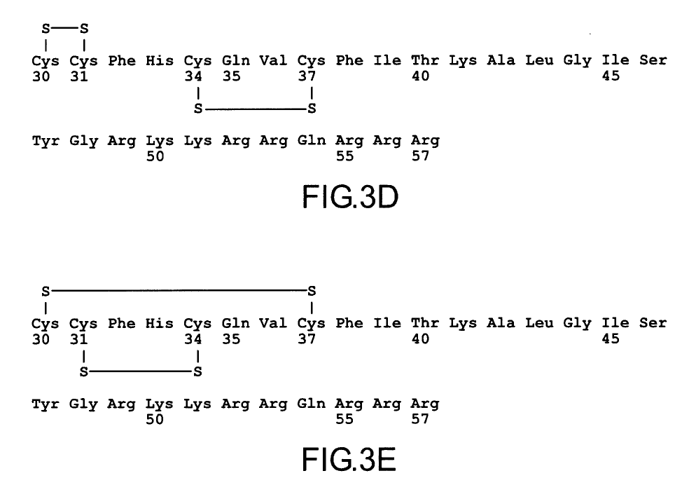

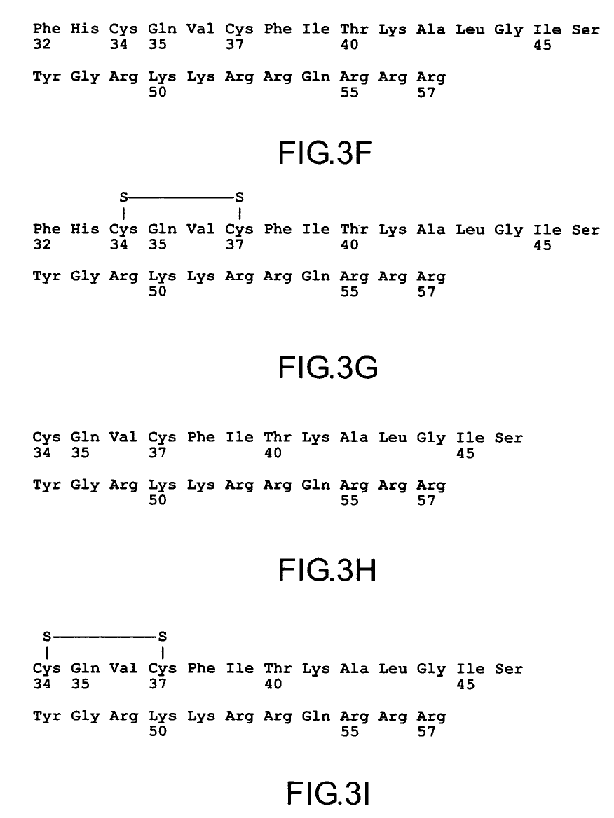

Now referring primarily to FIG. 3, the smaller linear peptide fragment HIV1-30-57 (SEQ ID. NO.: 38) as shown by FIG. 3A can be chemically synthesized in similar fashion as above-described and S-acetamidomethyl-L-cysteine (Cys-Acm) and S-Trt-L-cysteine can be incorporated in various combinations and permutations as residue 30, residue 31, residue 34 and as residue 37 to generate isomeric peptides of the disulfide bridge of HIV1 30-57 (SEQ ID. NO.: 38) including HIV1 30-57 Cys30Acm, Cys34Acm, (Cys31-Cys37) (SEQ ID NO.: 14) as shown by FIG. 3B; HIV1 30-57 (Cys30-Cys34)(Cys31-Cys37) (SEQ ID NO.: 16) as shown by FIG. 3C; HIV1 30-57 (Cys30-Cys31)(Cys34-Cys37) (SEQ ID. NO.: 27) as shown by FIG. 3D; and HIV1 30-57 (Cys30-Cys37)(Cys31-Cys34) (SEQ ID NO.: 26) as shown by FIG. 3E. As shown by FIGS. 3F and 3G, smaller peptide fragments HIV 32-57 (SEQ ID NO.: 28) along with HIV 32-57 (Cys34-Cys37) (SEQ ID NO.: 29) having a disulfide bridge between the two cysteine residues at positions 34 and 37, and as shown by FIGS. 3H and 3I, HIV 34-57 (SEQ ID NO.: 39) along with HIV 34-57 (Cys34-Cys37) (SEQ ID NO.: 40) having a disulfide bridge between the two cysteine residues at positions 34 and 37 can be similarly chemically synthesized as above-described using S-acetamidomethyl-L-cysteine (Cys-Acm) or S-Trt-L-cysteine residues to facilitate the formation of the disufide bridges.

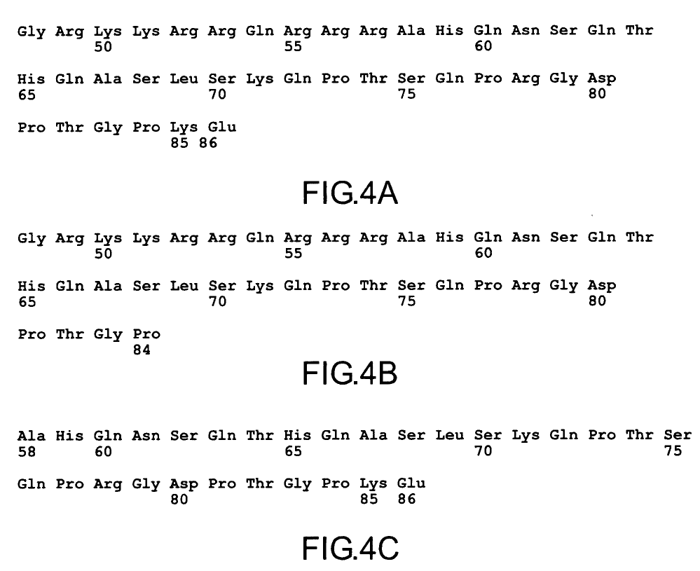

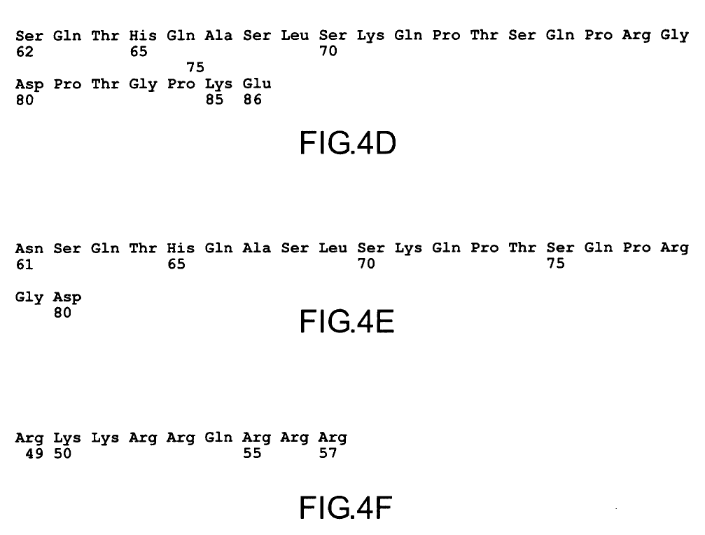

Now referring to FIG. 4, other peptide fragments of the C-terminal region of the HIV1 30-86 peptide including as a non-limiting example HIV1 48-86 (SEQ ID NO.: 19) as shown by FIG. 4A; HIV1 48-84 (SEQ ID NO.: 19) as shown by FIG. 4B; HIV1 58-86 (SEQ ID NO.: 37) as shown by FIG. 4C; HIV1 62-86 (SEQ ID NO.: 31) as shown by FIG. 4D; and HIV1 61-80 (SEQ ID NO.: 32) as shown by FIG. 4E and the peptide fragments of the C-terminal of the HIV1 30-57 peptide including as a non-limiting example the HIV1 49-57 Tat Region (SEQ ID NO.: 20) as shown by 4F can each be chemically synthesized as above-described.

The invention also encompasses additional polypeptides or peptide fragments as set forth in the sequence listing attached, hereby incorporated by reference, and polypeptides and peptide fragments of HIV1 Tat polypeptides as above-described which have substantially similar amino acid sequence and which are capable of or can be used for facilitating transfer of membrane impermeable compounds into the cell; mobilizing intracellular calcium ion including but not limited to calcium from the endoplasmic reticulum, tharsigargin-sensitive and tharsigargin-insensitives stores, acid filled calcium stores; activating calcium-induced calcium release; opening voltage-gated calcium channels; acting as HIV-1 tat antagonists; acting as calcium mobilization antagonists; treating vascular disorders, stroke, or hypertension; treating HIV-1 dementia, Alzheimer's Disease, or other dementia or neurological disorders; treating elevated or low calcium levels; or the like.

Silent substitutions of amino acid residues wherein the replacement of the residue with structurally or chemically similar residue(s) which do not significantly alter the structure, conformation, or activity of the polypeptide are intended to fall within the scope of the claims of this application including without limitation silent substitutions of amino acids of the purified polypeptides described above or set out in the Sequence Listing and further including instances in which one or more residues has been removed from either end or both ends, or from an internal region of the peptides (for example without limitation removal of one or more residues between position 41 and position 48 of the HIV1 32-62 peptide), or wherein one or more residues is added to either end or both ends, or to an internal location in either peptide (for example without limitation insertion of one or more residues between position 41 and position 48 of the HIV1 32-62 peptide). Additionally, purified polypeptides having chemical moieties or residues added for chemical or radiolabeling, such as, an added tyrosine for ¹²⁵iodine labeling are also understood to be encompassed by the invention. Similarly, the N-terminus of purified polypeptide encompassed by the invention can be prepared as amino, acetyl, formyl, or left with a residual FMOC or BOC group intact. As to certain other embodiments of the invention, the C-terminus was left bound to the resin, or cleaved to yield various C-terminal moieties, such as carboxyl or amide by choice of the corresponding BHA, PAM, or amide solid phase resin.

Similarly, as to the specific peptide sequences included in the sequence listing, or as described above, and specifically including without limitation SEQ ID NO: 3 and SEQ ID NO: 9 each can be used to model a numerous and wide variety of peptide analogs including those above-described and others, each peptide or peptide analog is intended to be included within the description of this application. Moreover, with respect to those specific peptides which contain a single cysteine residue or a plurality of cysteine residues in the linear sequence, the numerous and wide variety of molecular structures capable of being generated by forming one or a plurality of disulfide bridge within a single peptide or between a plurality of peptides is also intended to be encompassed by this description.

Importantly, because certain peptide fragments of the HIV 1 Tat polypeptides encompassed by the invention have between one and seven cysteine residues in their respective amino acid sequence these peptide fragments can be useful in generating numerous and varied peptide analogs which contain disulfide bridges as above-described or otherwise. Peptide fragments of the HIV 1 Tat polypeptides encompassed by the invention which contain certain disulfide bridges or combinations of disulfide bridges unexpectedly demonstrate similar or altered biological activity compared to the intact HIV 1 Tat polypeptides. As described below, the biological activity exhibited by the various peptide fragments of HIV 1 polypeptides, peptide fragments of HIV 1 polypeptides chemically synthesized, or peptide analogs thereof, can be greater or lesser than the intact HIV 1 Tat polypeptides and as such, the invention affords peptide fragments of HIV 1 Tat polypeptides which afford a graded range of biological activity.

To demonstrate biological activity of peptide fragments of HIV 1 polypeptides, peptide fragments of HIV 1 polypeptides chemically synthesized, or peptide analogs thereof, the mobilization of intracellular Ca²⁺ can be measured. Neurons can be isolated from cerebral cortex according to the protocols for postnatal dissociated nuerons as disclosed for example by Huettner, J. E. and Baughman, R. W., Primary Culture of Identified Neurons From the Visual Cortex of Postnatal Rats, Journal of Neurosciences 6; 3044-3060 (1986); Brewer, G. J., Isolation and Culture of Adult Rat Hippocampal Neurons, Journal of Neuroscience Methods, 71:143-155 (1997); and Brailoiu et al., NAADP Potentiates Neurite Outgrowth, Journal of Biological Chemistry (in press), each hereby incorporated by reference. Newborn Sprague-Dawley rates (about 1 day to about 4 days old) were killed by cervical dislocation. Cerebral cortex was removed and quickly immersed in ice-cold phosphate buffer solution. After removal of meninges, tissue was minced into about 1-millimeter blocks, incubated for about 45 minutes at 37° C. in Hanks balanced salt solution without Ca²⁺ and Mg²⁺ (Invitrogen, 1600 Faraday Avenue, Carlsbad, Calif. 92008) and supplemented with about 200 ug/mL penicillin, 0.1% EDTA and papain 0.15 mg/mL (Sigma-Aldrich, St. Louis, Mo.). In accordance with the procedure disclosed by Brewer, the tissue can be further dissociated by gentle mechanical trituration. After centrifugation at 500×g, cells can be re-suspended in fetal serum free media containing Neurobasal-A™ medium supplemented with 20 mM glutamine, 100 unites penicillin, 100 ug streptomycin, and B27 supplement all of which can be obtained for example from Invitrogen. The resulting cells can be plated at a low density of about 10⁴ on round glass coverslips in twenty four well plates. Neurons were cultrued at 37° C. in 95% oxygen and 5% carbon dioxide for about 3 days to about 5 days. The mitotic inhibitor, cytosine β-arabino furanosidde (about 1 μM) which can be obtained from Sigma-Aldrich can be added to cultured neurons to inhibit glial cell proliferation according to the procedure disclosed by Billingsley, M. L. and Mandel, H. G., Effects of DNA Synthesis Inhibitors on Post-Traumatic Glial Cell Proliferation, Journal of Pharmacology and Experimental Therapeutics, 222: 765-770 (1982).

Measurement of mobilized intracellular calcium ion (Ca²⁺) in dissociate cultured neurons in response to peptide fragments of HIV-1 Tat polypeptides can be performed as disclosed by Brailoiu E. et al. Neurons cultured about 24 hours on coverslips as above-described were loaded in HBBS with 5μ Fura-2 AM dye at room temperature (about 20° C.) for about 45 minutes in the dark, then washed three times with Fura-2 AM free buffer and allowed to incubate to allow de-esterification of the dye for about 45 minutes. Under these conditions, compartmentalization of the dye was minimal (about 9.2%±0.2% n=6) as judged from the ratio of fluorescence signals after selective permeabilization of the plasma membrane (10 μM β-escein) and full permeabilization of the cultured cells (60 μg/mL saponin). The coverslips were mounted in a custom designed bath on the stage of a S300 Axiovert Nikon inverted microscope equipped with a C&L Instruments fluorimeter as described by Brailoiu et al. The Fura-2 fluorescent signal was calibrated by successive addition of 20 μg/mL digitonin, 20 mM EDTA, and 0.5 mM MnCl₂. Ca²⁺ values were then obtained using the procedures and equation described by Grynkiewicz, G., Poenie, M., and Tsien, R. Y. A New Generation of Ca²⁺ Indicators With Greatly Improved Fluorescence Properties, J. Biol. Chem., 260:3440-3450 (1989)

Now referring to Table 1, measurement of intracellular calcium ion (Ca²⁺) in dissociate cultured neurons shows that Ca²⁺ can be mobilized in response to peptide fragments of HIV-1 Tat polypeptides and analogs thereof not heretofore described.

TABLE 1
Effect of Peptide Fragments of HIV1-Tat Polypeptides on [Ca²⁺] Response in
Dissociate and Cultured Cortical Neurons

| HIV1 Tat Polypeptide Fragment | code | SEQ NO. | [Ca²⁺] nM | n |
|---|---|---|---|---|
| 30-47 (Cys 30-Cys34)(Cys 31-Cys37) | 075-20 | 35 | No Response | 6 |
| 30-57 Cys30Acm, Cys34Acm, (Cys3l-Cys37) | — | 14 | 197 ± 35 | 10 |
| 30-57 (Cys30-Cys34)(Cys31-Cys37) | 075-21 | 16 | 528 ± 60 | 10 |
| 30-57 (Cys30-Cys31)(Cys34-Cys37) | 075-24 | 27 | 258 ± 54 | 10 |
| 30-57 (Cys30-Cys37)(Cys31-Cys34) | 075-23 | 26 | 197 ± 46 | 10 |
| 30-86 (Cys30-Cys34)(Cys31-Cys37) | 075-18 | 9 | 390 ± 21 | 9 |
| 30-86 Cys30Acm, Cys34Acm (Cys31-Cys37) | — | 13 | No Response | 5 |
| 32-62 (Cys34-Cys37) | 018-51 | 3 | 752 ± 59 | 8 |
| 41-60 | 018-53 | 30 | 66 ± 13 | 6 |
| 48-84 | — | 19 | 80 ± 28 | 7 |
| 49-57 | 075-25 | 20 | No Response | 8 |
| 58-86 | 075-19 | 37 | 54 ± 28 | 7 |
| 61-80 | 018-54 | 32 | 56 ± 20 | 6 |
| 62-86 | 075-22 | 31 | No Response | 5 |

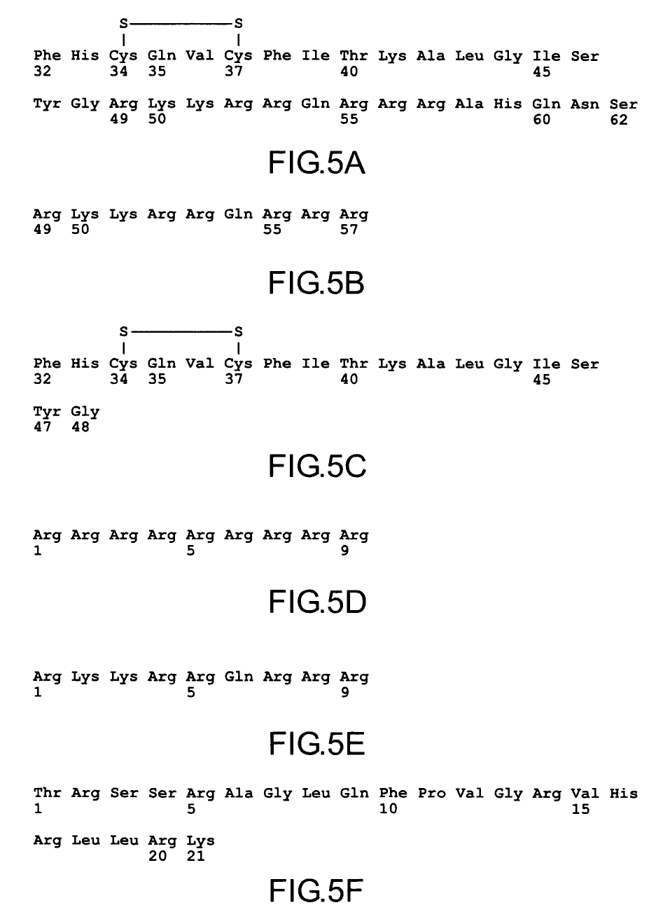

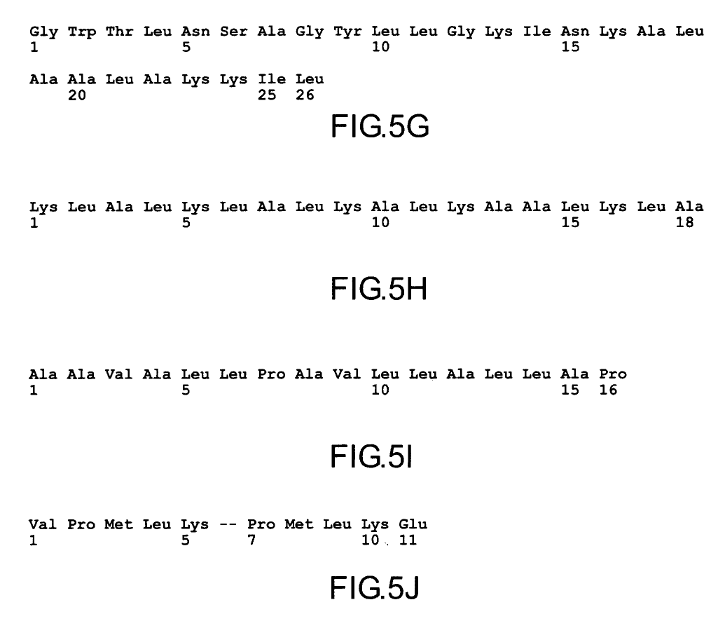

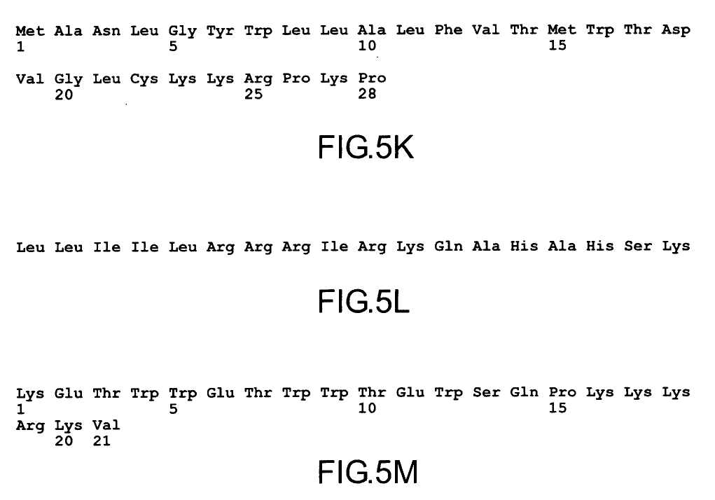

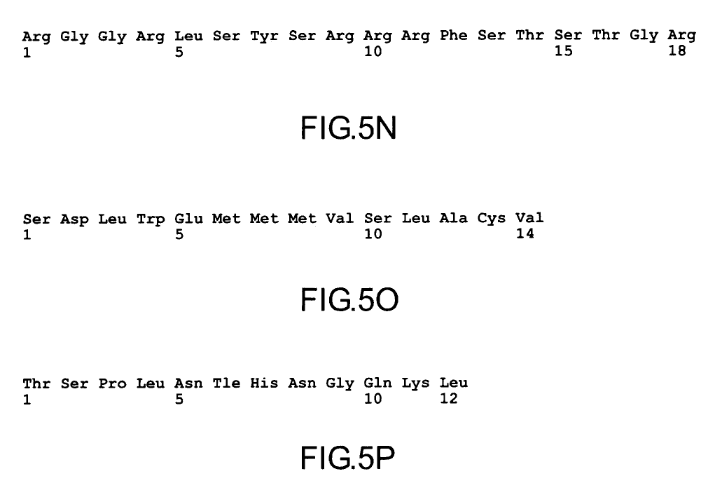

Now referring primarily to FIGS. 5A, 5B and 5C, the mobilization of intracellular calcium ion (Ca²⁺) in dissociate cultured neurons in response to the family of peptide fragments and peptide analogs of the invention shows that each peptide fragment or peptide fragment analog (FIG. 5A provides a non-limiting example) contains at least a first peptide region (FIG. 5B provides a non-limiting example) which functions to transfer itself along with otherwise membrane impermeable compounds into the cell and a second peptide region (FIG. 5C provides a non-limiting example) which can function in the cell to regulate the variety of intercellular responses associated with intact HIV 1 Tat polypeptides such as the production of interferon and the subsequent cascade of events leading to inhibition of protein synthesis; binding of a variety of cellular factors, including, but not limited to HIV long terminal repeat (LTR) RNA trans-activation response (TAR) element region, ATPase and DNA helicase, 36-kDA nuclear factor, as well as the transcriptional factors FFIID and Sp11; along with affecting neurotransmitter release including, but not limited to, the release of acetylcholine and inducing neurological impairments and neurotoxicity by mechanisms involving Ca²⁺ homeostasis after binding and depolarizing neuronal membranes.

Understandably the first peptide region of the invention can comprise the Tat transduction domain (RKKRRQRRR) HIV 1 49-57 (SEQ ID NO.: 20), as shown by FIG. 5B, which allows transfer of the second portion of the peptide fragments and peptide analogs encompassed by the invention into the cell; however, it is not intended that the first peptide region of the invention be limited solely to the use of Tat transduction domain HIV 49-57 for transfer of the second portion of the peptide across the cell membrane, and a numerous and wide variety of other peptides sequences can be utilized as the first peptide region to transfer embodiments of the invention across the cell membrane. These alternate peptide structures include, but are not limited to, other arginine-rich peptides such as polyarginine and in particular RRRRRRRRR as shown by FIG. 5D or other cell permeable peptides such as Penetratin as shown by FIG. 5E or Buforin II as shown by FIG. 5F or other synthesized cell permeable peptides such as Transportan, MAP, K-FGF, Ku70, Prion, pVEC, Pep-1, SyB1 as shown by FIGS. 5G through 5N or other phage display cell permeable peptides such as Pep-7 and HN-1, as shown by FIGS. 5O and 5P or similar peptides which allow transfer of the second region of the peptide to the target binding site. See also: Joliot, A. and Prochiantz, A., Transduction Peptides: From Technology to Physiology, Nature Cell Biology, Vol. 6, No. 3, March 2004, hereby incorporated by reference.

Again referring to FIG. 5 and Table 1 it can be understood that the invention can comprise without more a second peptide region which functions in the cell to regulate the variety of intercellular responses as above described. The second peptide region can comprise any of the above-described peptide fragments, peptide fragment analogs, or peptide fragments or peptide fragment analogs which further provide at least one disulfide bridge between two cysteine residues less the first peptide region or other peptide sequence responsible for transfer of the second peptide region into the cell. Specifically, the second peptide region can comprise SEQ ID NO.: 22, HIV 1 30-47; SEQ ID NO.: 35, HIV 1 30-47 (Cys 30-Cys34)(Cys 31-Cys37); SEQ ID. NO.: 28, HIV1 32-47; SEQ ID. NO.: 29, HIV1 32-47 (Cys34-Cys37); SEQ ID. NO.: 39, HIV1 34-47; SEQ ID. NO: 40, HIV1 34-47 (Cys34-Cys37) or analogs thereof, or can comprise SEQ ID NO.: 38, HIV 30-57; SEQ ID NO.: 16, HIV 1 30-57 (Cys30-Cys34)(Cys31-Cys37); SEQ ID. NO: 27, HIV1 30-57 (Cys30-Cys31)(Cys34-Cys37); SEQ ID. NO.: 26, HIV1 30-57 (Cys30-Cys37)(Cys31-Cys34) or analogs thereof, less the Tat transduction domain. Embodiments of the second peptide region can also include SEQ ID NO.: 7, HIV 1 30-86; SEQ ID NO.: 9, HIV 1 30-86 (Cys30-Cys34)(Cys31-Cys37); SEQ ID NO: 3, HIV 1 32-62 (Cys34-Cys37) or analogs thereof, less the Tat transduction domain along with any additional C-terminal residues of the HIV 1 Tat polypeptide.

The invention can further encompass peptides which include a first peptide region coupled to a second peptide region which can function in the cell to regulate any one of the variety of intercellular responses above-described, and specifically includes without limitation the examples of the invention included in Table 1 which can mobilize intracellular calcium ion (Ca²⁺), and specifically includes without limitation each of the peptide structures described by SEQ ID NO.: 14, SEQ ID NO.: 16, SEQ ID NO.: 27, SEQ ID NO.: 26, SEQ ID NO.: 9, SEQ ID NO.: 3 SEQ ID NO.: 28, SEQ ID NO.: 29, SEQ ID NO.: 38, SEQ ID NO.: 39, SEQ ID NO.: 40, along with any analogs thereof.

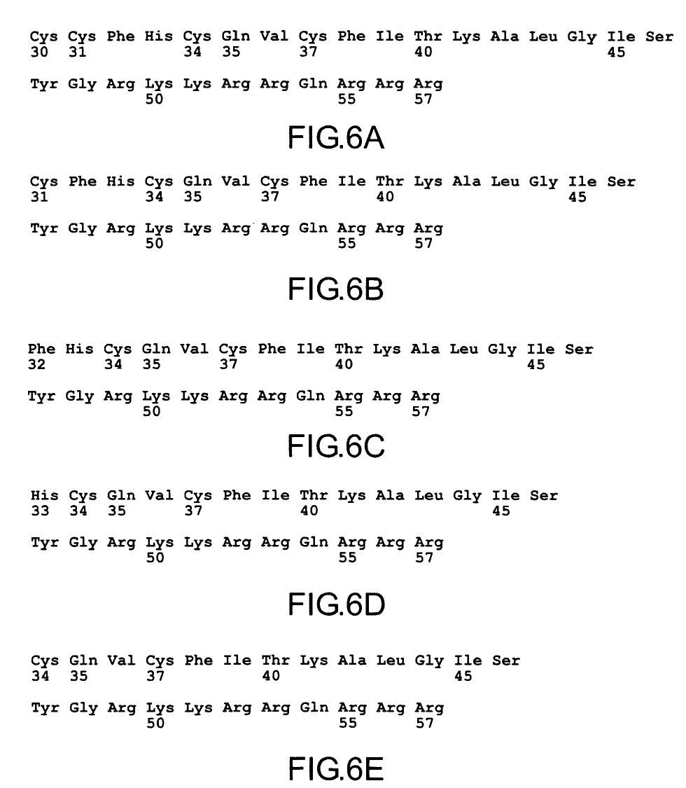

Now referring to FIG. 6, the invention further includes a heretofore undisclosed isolated biologically active regions of the HIV1 Tat polypeptides which include each of the contiguous amino acid sequences beginning with residue 30 and terminating with residue 57 of the HIV1 Tat polypeptide (FIG. 6A), the contiguous amino acid sequence beginning with residue 31 and terminating with residue 57 of the HIV1 Tat polypeptides (FIG. 6B), the contiguous amino acid sequence beginning with residue 32 and terminating with residue 57 of the HIV1 Tat polypeptides (FIG. 6C), the contiguous amino acid sequence beginning with residue 33 and terminating with residue 57 of the HIV1 Tat polypeptides (FIG. 6D), or the contiguous amino acid sequence beginning with residue 34 and terminating with residue 57 of the HIV1 Tat polypeptides (FIG. 6E), (each of such biologically active regions further including a family of molecular structures distinguished by the inclusion of various disulfide bridges that can be formed between the cysteine residues encompassed by each such biologically active region, or lack thereof, that when isolated by enzymatic or chemical excision from the intact HIV1 Tat polypeptide, or in the alternative isolated by chemical synthesis of the amino acid sequence as above-described, and in either event purified, provides a molecular structure having the capacity to travel across the cell membrane and target binding sites within the cell to regulate cell processes such as the production of interferon and the subsequent cascade of events leading to inhibition of protein synthesis; binding of a variety of cellular factors, including, but not limited to HIV long terminal repeat (LTR) RNA trans-activation response (TAR) element region, ATPase and DNA helicase, 36-kDA nuclear factor, as well as the transcriptional factors FFIID and Sp11; along with affecting neurotransmitter release including, but not limited to, the release of acetylcholine and inducing neurological impairments and neurotoxicity by mechanisms involving Ca²⁺ homeostasis after binding and depolarizing neuronal membranes.

The particular molecular structures, polypeptides, peptides or peptide analogs of the invention disclosed by the description or shown in the figures accompanying this application are not intended to be limiting, but rather exemplary of the numerous and varied molecular structures, polypeptides, peptides, peptide analogs, or equivalents thereof generically encompassed by the invention. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “peptide binder” should be understood to encompass disclosure of the act of “peptide binding”—whether explicitly discussed or not—and, conversely, were there effectively disclosure of the act of “peptide binding”, such a disclosure should be understood to encompass disclosure of a “peptide binder” and even a “means for binding.” Such alternative terms for each element or step are to be understood to be explicitly included in the description.

In addition, as to each term used it should be understood that unless its utilization in this application is inconsistent with such interpretation, common dictionary definitions should be understood to included in the description for each term as contained in the Random House Webster's Unabridged Dictionary, second edition, each definition hereby incorporated by reference.

Thus, the applicant(s) should be understood to claim at least: i) each of the peptides herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The claims set forth in this specification are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

The claims set forth below are intended describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

I claim:

1. A purified polypeptide the amino acid sequence of which comprises SEQ ID NO: 2.

2. The purified polypeptide of claim 1 which consists of SEQ ID NO: 2.

3. The purified polypeptide of claim 2, wherein said purified polypeptide has an amide C-terminus.

4. The purified polypeptide of claim 2, wherein said purified polypeptide has an acetyl N-terminus.

5. The purified polypeptide of claim 4, wherein said purified polypeptide has an acetyl N-terminus.

6. The purified polypeptide of claim 1, wherein said purified polypeptide has a disulfide bridge between residues 3 and 6.

7. The purified polypeptide of claim 2, wherein said purified polypeptide has a disulfide bridge between residues 3 and 6.

8. The purified polypeptide of claim 7, wherein said purified polypeptide has an amide C-terminus.

9. The purified polypeptide of claim 7, wherein said purified polypeptide has an acetyl N-terminus.

10. The purified polypeptide of claim 6, wherein said purified polypeptide has an amide acetyl N-terminus.

11. The purified polypeptide of claim 6, wherein said purified polypeptide has a sequence identity with the consecutive amino acid sequence selected from the group of: at least 80%, at least 85%, at least 90%, at least 95%, and at least 98%.

12. The purified polypeptide of claim 6, wherein said purified polypeptide has at least one conservative amino acid substitution.

13. The purified polypeptide of claim 7, comprising at least 30 contiguous residues.

14. The purified polypeptide of claim 13, comprising at least 29 contiguous residues.

15. The purified polypeptide of claim 14, comprising at least 29 contiguous residues including a disulfide bridge between residue 3 and residue 6.

16. The purified polypeptide of claim 15, comprising at least 24 contiguous residues including the disulfide bridge between residue 3 and residue 6.

17. The purified polypeptide of claim 16, comprising at least 15 contiguous residues including the disulfide bridge between residue 3 and residue 6.

18. The purified polypeptide of claim 17, comprising at least 10 contiguous residues including the disulfide bridge between residue 3 and residue 6.

19. The purified polypeptide of claim 15, comprising the 29 contiguous residues between residue 3 and residue 31 including the disulfide bridge between residue 3 and residue 6 consisting of SEQ ID NO.: 3.

20. The purified polypeptide of claim 16, comprising the 24 contiguous residues between residue 3 and residue 26 including the disulfide bridge between residue 3 and residue 6 consisting of SEQ ID NO.: 40.

21. A purified polypeptide which mobilizes intracellular calcium release the amino acid sequence of which is selected from the group consisting of: SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33, SEQ ID NO. 34, SEQ ID NO. 35, SEQ ID NO. 36, SEQ ID NO. 37, SEQ ID NO. 38, SEQ ID NO. 39, and SEQ ID NO. 40.

22. A purified poly peptide the amino acid sequence of which consists of SEQ ID NO: 9.


SEQUENCE LISTING (40 sequences)

SEQ ID NO: 1 - protein, 86 aa, Human immunodeficiency virus type 1
MEPVDPRLEPWKHPGSQPKTACTNCYCKKCCFHCQVCFITKALGISYGRKKRRQRRRAHQNSQTHQASLSKQPTSQPRGDPTGPKE

SEQ ID NO: 2 - protein, 31 aa, Human immunodeficiency virus type 1
FHCQVCFITKALGISYGRKKRRQRRRAHQNS

SEQ ID NO: 3 - protein, 31 aa, Human immunodeficiency virus type 1; identical to SEQ ID NO: 2
  disulfide (3)..(6)

SEQ ID NO: 4 - protein, 31 aa, Human immunodeficiency virus type 1; identical to SEQ ID NO: 2
  disulfide (3)..(6)
  amide (31)..(31)

SEQ ID NO: 5 - protein, 31 aa, Human immunodeficiency virus type 1; identical to SEQ ID NO: 2
  acetyl (1)..(1)
  disulfide (3)..(6)
  amide (31)..(31)

SEQ ID NO: 6 - protein, 29 aa, Human immunodeficiency virus type 1
MEPVDPRLEPWKHPGSQPKTACTNCYCKK

SEQ ID NO: 7 - protein, 57 aa, Human immunodeficiency virus type 1
CCFHCQVCFITKALGISYGRKKRRQRRRAHQNSQTHQASLSKQPTSQPRGDPTGPKE

SEQ ID NO: 8 - protein, 57 aa, Human immunodeficiency virus type 1; identical to SEQ ID NO: 7
  disulfide (2)..(8)

SEQ ID NO: 9 - protein, 57 aa, Human immunodeficiency virus type 1; identical to SEQ ID NO: 7
  disulfide (1)..(5)
  disulfide (2)..(8)

SEQ ID NO: 10 - protein, 57 aa, Human immunodeficiency virus type 1; identical to SEQ ID NO: 7
  disulfide (1)..(5)
  disulfide (2)..(8)
  amide (57)..(57)

SEQ ID NO: 11 - protein, 57 aa, Human immunodeficiency virus type 1; identical to SEQ ID NO: 7
  disulfide (1)..(5)
  acetyl (1)..(1)
  disulfide (2)..(8)
  amide (57)..(57)

SEQ ID NO: 12 - protein, 57 aa, Human immunodeficiency virus type 1; identical to SEQ ID NO: 7
  cys-acm (1)..(1): cysteine having side chain protected by covalent linkage to an acetamidomethyl group

SEQ ID NO: 13 - protein, 57 aa, Human immunodeficiency virus type 1; identical to SEQ ID NO: 7
  cys-acm (1)..(1): cysteine residues having the sidechain sulfur atom protected by covalent linkage to an acetamidomethyl group

SEQ ID NO: 14 - protein, 28 aa, Human immunodeficiency virus type 1
  cys-acm (1)..(1): cysteine residue having the sidechain sulfur atom protected by covalent linkage to an acetamidomethyl group
CCFHCQVCFITKALGISYGRKKRRQRRR

SEQ ID NO: 15 - protein, 28 aa, Human immunodeficiency virus type 1
  disulfide (1)..(5)
  cys-acm (2)..(2): represent cysteine residue having the sidechain sulfur atom protected by covalent linkage to an acetamidomethyl group
CCFHCQVCFITKALGISYGRKKRRQRRR

SEQ ID NO: 16 - protein, 28 aa, Human immunodeficiency virus type 1
  disulfide (1)..(5)
  disulfide (2)..(8)
CCFHCQVCFITKALGISYGRKKRRQRRR

SEQ ID NO: 17 - protein, 28 aa, Human immunodeficiency virus type 1
  disulfide (1)..(5)
  disulfide (2)..(8)
  amide (28)..(28): C-terminal amide
CCFHCQVCFITKALGISYGRKKRRQRRR

SEQ ID NO: 18 - protein, 28 aa, Human immunodeficiency virus type 1
  disulfide (1)..(5)
  acetyl (1)..(1)
  disulfide (2)..(8)
  amide (28)..(28)
CCFHCQVCFITKALGISYGRKKRRQRRR

SEQ ID NO: 19 - protein, 37 aa, Human immunodeficiency virus type 1
GRKKRRQRRRAHQNSQTHQASLSKQPTSQPRGDPTGP

SEQ ID NO: 20 - protein, 9 aa, Human immunodeficiency virus type 1
RKKRRQRRR

SEQ ID NO: 21 - protein, 18 aa, Human immunodeficiency virus type 1
CCFHCQVCFITKALGISY

SEQ ID NO: 22 - protein, 18 aa, Human immunodeficiency virus type 1
  disulfide (1)..(5)
  disulfide (2)..(8)
  amide (18)..(18)
CCFHCQVCFITKALGISY

SEQ ID NO: 23 - protein, 28 aa, Human immunodeficiency virus type 1
  disulfide (1)..(2)
CCFHCQVCFITKALGISYGRKKRRQRRR

SEQ ID NO: 24 - protein, 28 aa, Human immunodeficiency virus type 1
  disulfide (5)..(8)
CCFHCQVCFITKALGISYGRKKRRQRRR

SEQ ID NO: 25 - protein, 28 aa, Human immunodeficiency virus type 1
  disulfide (1)..(8)
CCFHCQVCFITKALGISYGRKKRRQRRR

SEQ ID NO: 26 - protein, 28 aa, Human immunodeficiency virus type 1
  disulfide (1)..(8)
  disulfide (2)..(5)
CCFHCQVCFITKALGISYGRKKRRQRRR

SEQ ID NO: 27 - protein, 28 aa, Human immunodeficiency virus type 1
  disulfide (1)..(2)
  disulfide (5)..(8)
CCFHCQVCFITKALGISYGRKKRRQRRR

SEQ ID NO: 28 - protein, 26 aa, Human immunodeficiency virus type 1
FHCQVCFITKALGISYGRKKRRQRRR

SEQ ID NO: 29 - protein, 26 aa, Human immunodeficiency virus type 1
  disulfide (3)..(6)
FHCQVCFITKALGISYGRKKRRQRRR

SEQ ID NO: 30 - protein, 20 aa, Human immunodeficiency virus type 1
KALGISYGRKKRRQRRRAHQ

SEQ ID NO: 31 - protein, 25 aa, Human immunodeficiency virus type 1
SQTHQASLSKQPTSQPRGDPTGPKE

SEQ ID NO: 32 - protein, 20 aa, Human immunodeficiency virus type 1
NSQTHQASLSKQPTSQPRGD

SEQ ID NO: 33 - protein, 18 aa, Human immunodeficiency virus type 1
CCFHCQVCFITKALGISY

SEQ ID NO: 34 - protein, 18 aa, Human immunodeficiency virus type 1
  amide (18)..(18)
CCFHCQVCFITKALGISY

SEQ ID NO: 35 - protein, 18 aa, Human immunodeficiency virus type 1
  disulfide (1)..(5)
  disulfide (2)..(8)
CCFHCQVCFITKALGISY

SEQ ID NO: 36 - protein, 36 aa, Human immunodeficiency virus type 1
RKKRRQRRRAHQNSQTHQASLSKQPTSQPRGDPTGP

SEQ ID NO: 37 - protein, 29 aa, Human immunodeficiency virus type 1
AHQNSQTHQASLSKQPTSQPRGDPTGPKE

SEQ ID NO: 38 - protein, 28 aa, Human immunodeficiency virus type 1
CCFHCQVCFITKALGISYGRKKRRQRRR

SEQ ID NO: 39 - protein, 14 aa, Human immunodeficiency virus type 1
CQVCFITKALGISY

SEQ ID NO: 40 - protein, 24 aa, Human immunodeficiency virus type 1
  disulfide (3)..(6)
CQVCFITKALGISYGRKKRRQRRR